(19) United States
(12) Patent Application Publication
(10) Pub. No.: US 2022/0386860 A1
(43) Pub. Date: Dec. 8, 2022

(54) DENTAL MIRROR SYSTEM

(71) Applicant: KMIHH KII.AM LTD, Kdumim (IL)

(72) Inventors: Or RAMOT, Oranit (IL); Arye SHAHAR, Palm Coast, FL (US)

(21) Appl. No.: 17/777,123

(22) PCT Filed: May 20, 2021
(86) PCT No.: PCT/IB2020/060734

§ 371 Date: May 16, 2022

Related U.S. Application Data
(60) Provisional application No. 62/936,531, filed on Nov. 17, 2019.

(51) Int. Cl. A61B 1/253

(52) U.S. Cl. CPC A61B 1/253

(57) ABSTRACT

A dental minor system has an axis and is arranged to detachably receive minor heads at its distal axial side. The system includes a threaded internal passage and an airflow nozzle. The threaded internal passage is arranged to receive each time a threaded member of one minor head and the airflow nozzle is rotatable about the axis in order to align an airflow emitted by the nozzle against reflecting surface of the mirror head.

### TECHNICAL FIELD

Embodiments of the invention relate to a dental mirror system, in particular for housing replaceable mirrors.

### BACKGROUND

Mirrors assist in dentistry to examine teeth in areas of the mouth that are difficult to access and view. They provide indirect vision to the dentist by reflecting light onto target areas in the mouth, in particular teeth, while possibly also assisting in retraction of soft tissues obstructing the required views.

Mirrors used in dentistry can come in the form of mirror heads. A mirror head typically includes a thread for screwing it onto a hand-piece that the dentist uses for inspecting the mouth's interior. Various types of mirror heads exist, for example for inspecting a rear or front area of the mouth, for magnifying (etc.); and a dentist typically has several mirror head types in his clinic that he/she is used to working with.

When used in a patient's mouth, fog may develop on a dental mirror as a patient for example inhales and exhales. Such condensation that is formed can distort of block a view of a tooth being inspected. As a result, certain dental mirror systems provide an air flow that is directed to the surface of the mirror in order to vaporize such condensation being formed.

U.S. Pat. No. 9,629,533 for example describes a dental mirror instrument that has an airflow conduit that extend along the length of an elongated member to a mirror portion. An airflow exiting from the airflow conduit may be directed over the mirror surface to remove fluids and debris.

### SUMMARY

The following embodiments and aspects thereof are described and illustrated in conjunction with systems, tools and methods which are meant to be exemplary and illustrative, not limiting in scope.

In an embodiment there is provided a dental mirror system extending along an axis and arranged to detachably receive mirror heads at its distal axial side.

Such mirror heads may typically be standardized mirror heads normally available in a dental clinic, wherein embodiments the dental system of the invention have been suited to receive such mirror heads while increasing utility available to a dentist using such mirrors—such as in provision of suitable airflow to remove condensation formed on the mirrors by aligning and/or regulating the airflow emitted against the mirrors.

In an embodiment, the system comprising a threaded internal passage and an airflow nozzle, the threaded internal passage being arranged to receive each time a threaded member of one mirror head and the airflow nozzle being rotatable about the axis in order to permit alignment of airflow emitted by the nozzle against a reflecting surface of the mirror head.

In other embodiments there is provided a method for aligning an airflow to a dental mirror comprising the steps of: providing a mirror dental system extending along an axis and comprising a threaded internal passage and an airflow nozzle, attaching a mirror head to the system by screwing a threaded stem of the mirror head into the internal passage until it is firmly secured therein, and rotating the airflow nozzle about the system's axis in order to align an airflow emitted by the nozzle against a reflecting surface of the mirror head.

In yet a further embodiment and/or aspect there is provided a method for regulating an airflow against a dental mirror comprising the steps of: providing a dental mirror system having an axis and comprising a handle, a shaft located axially distal to the handle and an airflow passage extending axially through the handle and shaft, wherein the handle comprises a regulating member and the shaft being arranged for attachment of mirror heads at its distal axial side, wherein rotating the handle relative to the shaft is arranged to regulate the flow of air/gas passing through the system and emitted against a reflecting surface of a mirror head attached to the system.

And in yet a further embodiment and/or aspect there is provided a dental mirror system extending along an axis and arranged to detachably receive mirror heads at its distal axial side, the system comprising a metallic insert comprising an internal thread for receiving corresponding threads of mirror heads that are screwed therein, wherein least a portion of the system is molded over the insert.

In addition to the exemplary aspects and embodiments described above, further aspects and embodiments will become apparent by reference to the figures and by study of the following detailed descriptions.

### BRIEF DESCRIPTION OF THE FIGURES

Exemplary embodiments are illustrated in referenced figures. It is intended that the embodiments and figures disclosed herein are to be considered illustrative, rather than restrictive. The invention, however, both as to organization and method of operation, together with objects, features, and advantages thereof, may best be understood by reference to the following detailed description when read with the accompanying figures, in which:

FIG. 1A schematically shows a dental mirror system in accordance with an embodiment of the present invention including a mirror head fitted at its front side in alignment with an airflow nozzle of the system;

FIG. 1B schematically shows a partial exploded view of the dental mirror system of FIG. 1 illustrating a sub-division into proximal and distal portions;

FIGS. 2A and 2B schematically show further exploded views of the proximal and distal portions, respectively;

FIGS. 3A and 3B schematically show a dental mirror system embodiment generally similar to those in former embodiments in airflow miss-aligned and aligned states, respectively; and

FIGS. 4A and 4B schematically show a dental mirror system in accordance with a further embodiment of the present invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated within the figures to indicate like elements.

### DETAILED DESCRIPTION

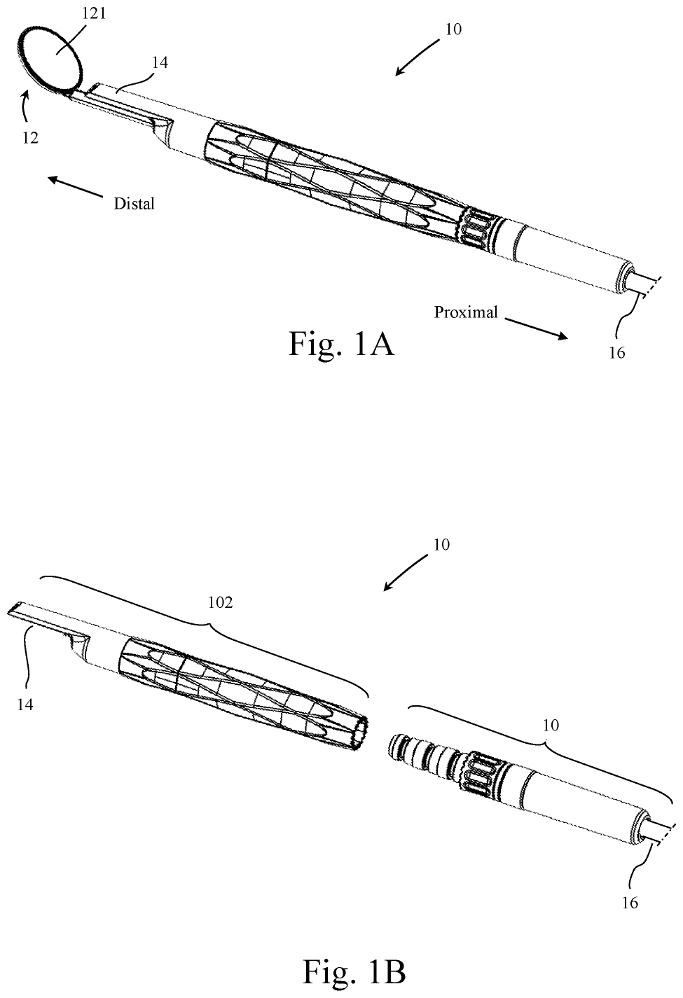

Attention is first drawn to FIGS. 1A and 1B schematically illustrating an embodiment of a dental mirror system 10 that can be fitted with a mirror head 12 (see FIG. 1A) at its distal front side. Mirror system 10 here includes an airflow nozzle 14 that in FIG. 1A is seen aligned to emit an airflow at a reflecting surface 121 of the mirror in order to vaporize any fog or condensation that may be formed on this surface e.g. during use of the system by a dentist for inspecting teeth within a patient's mouth.

Mirror head 12 may be of any typical type normally available in a dental clinic. For example, mirror head 12 may be of types used for inspecting rear or front areas of the mouth, for magnifying (etc.). Such mirror heads 12 typically include a thread at their proximal side for being secured into hand-piece devices normally available in dental clinics. Such threads of typically available mirror heads may be defined according to standards (e.g. ISO 9873—see third edition 2017-04 titled “Dentistry—Intra-oral mirrors”).

In accordance with an aspect of the present invention, a dentist thus may threadingly engage and/or disengage standardized mirror heads at the distal side of a dental mirror system of various embodiments of the invention according to his professional needs—as he would normally do when using such mirror heads with hand-piece devices normally available in dental clinics.

FIG. 1B shows dental mirror system 10 being formed from a first proximal portion 101 and a second distal portion 102 that may be axially joined together to form dental mirror system 10. Second distal portion 102 may include airflow nozzle 14 at its distal side and first proximal portion 101 may include an incoming conduit 16 at its proximal side for communicating an incoming air flow downstream and through dental system to be emitted at airflow nozzle 14. Incoming conduit 16 may be connected at its upstream side (not shown) to any suitable means available in a dental clinic providing pressurized air or gas.

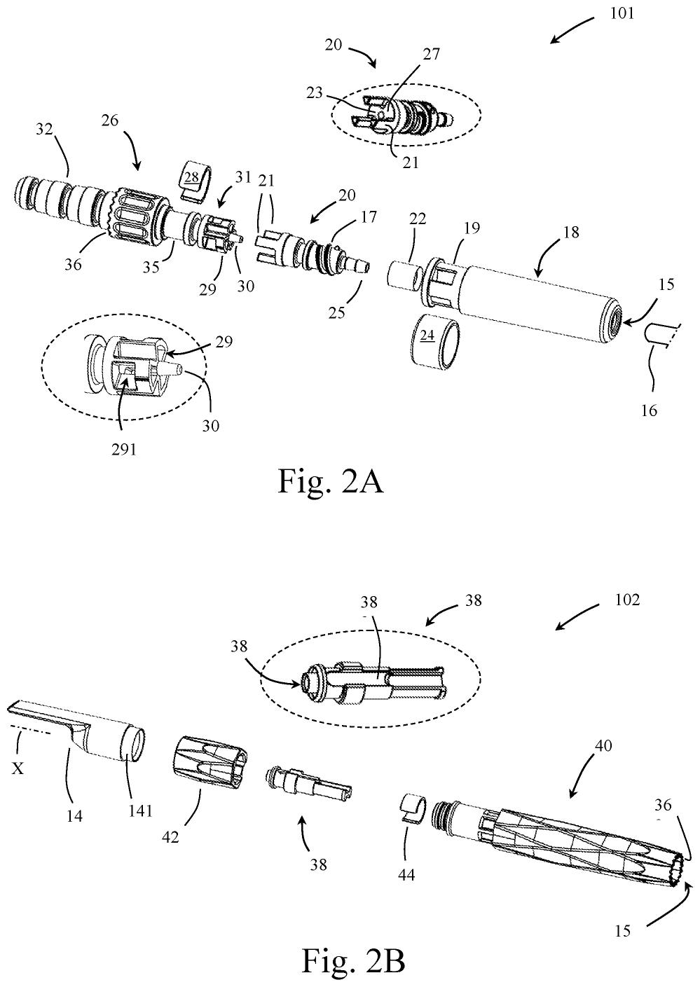

Attention is drawn to FIG. 2A that provides an exploded view of first proximal portion 101. Starting from the right hand proximal side of the figure, first proximal portion 101 can be seen including a handle 18, a regulator base 20 and a cylindrical ring 22. In an assembled state, regulator base 20 and cylindrical ring 22 are located within a through-going internal passage 15 of the handle.

Assembly of these parts may include first urging incoming conduit 16 to pass through internal passage 15 to then connect to a rear facing nipple 25 of base 20 with cylindrical ring 22 being arranged to surround this area of connection in order to secure it in place.

Then regulator base 20 may be inserted to within internal passage 15 to bring a thread 17 thereof into threading engagement with an internal thread (not shown) formed in passage 15. A cylindrical flange 20 may be fitted onto a recess 19 formed at a forward distal side of the handle's outer face.

Regulator base 20 in this example include arms 21 at its distal forward side formed about a longitudinal axis of the base with spacing's there between. The arms 21 project forwardly above a forward facing roof 27 of the base, and base 20 includes an aperture 23 where an internal lumen extending from nipple 25 and passing through base 20 opens out at roof 27. See, inter alia, roof 27 and aperture 23 in the upper section of regulator base 20 that is provided in this figure.

First proximal portion 101 includes in addition a regulator head 26 and a snap member 28. Regulator head 26 has a rear proximal anchor 31 with spaced apart pegs 29 formed about a longitudinal axis of the head. As seen in the section at the lower side of the figure, each peg 29 has internal passages 291 that provide fluid communication with an internal through-going passage that extends axially through regulator head 26.

Regulator head 26 in addition has a rear facing pin 30 at its rear proximal side that projects rearward away from anchor 31. Snap member 28 may be arranged to snap into a recess 35 located forward to anchor 31 and regulator head 26 has a forward coupler 32 that projects in the distal direction above a peripheral wavy shoulder 361 that faces the forward and distal direction.

In the assembled state of first proximal portion 101, the pegs 29 of regulator head 26 are arranged to mesh with the arms 21 of regulator base 20 (e.g., by placing pegs 29 in between arms 21)—and snap member 28 is arranged to engage with the internal passage 15 of handle 18 to thereby keep regulator head 26 fixed to handle 18 and snuggly received within internal passage 15 (together, inter alia, with regulator base 20 being accordingly engaged therewith via the pegs and arms).

Attention is drawn to FIG. 2B that provides an exploded view of second distal portion 102. Second distal portion 102 includes an internal insert 38, preferably made of metallic material (e.g. aluminum). Insert 38 includes a threaded internal passage 381 that extends at least along a portion of its longitudinal extension and is open to receive incoming screw heads from its distal side. Insert 38 in addition includes longitudinal extending by-pass passages 382 formed along its exterior face. See, inter alia, passages 381, 382 at the upper enlarged section in this figure.

Second distal portion 102 includes in addition a shaft 40 that is preferably molded over insert 38—so that insert 38 is in fact snuggly received integrally within shaft 40 possibly within a through-going internal passage 150 of the shaft. Shaft 40 includes a peripheral wavy shoulder 362 at its proximal end that faces the proximal direction and passage 150 opens out of the shaft at shoulder 362. Second distal portion 102 in addition includes a cylindrical flange 42, airflow nozzle 14 and a snap member 44.

Assembly of second distal portion 102 may be accomplished by coupling airflow nozzle 14 to the distal side of shaft 40 using snap member 44, while flange 42 is located there-between. Flange 42, preferably made from silicon material or the like, may be arranged to press against a proximal shoulder 141 of airflow flow nozzle 14 in an assembled state of second distal portion 102—in order to apply frictional forces against airflow nozzle 14 as it may be urged to rotate about a longitudinal axis X of distal portion 102 (where axis X is in fact also a longitudinal axis of dental system 10).

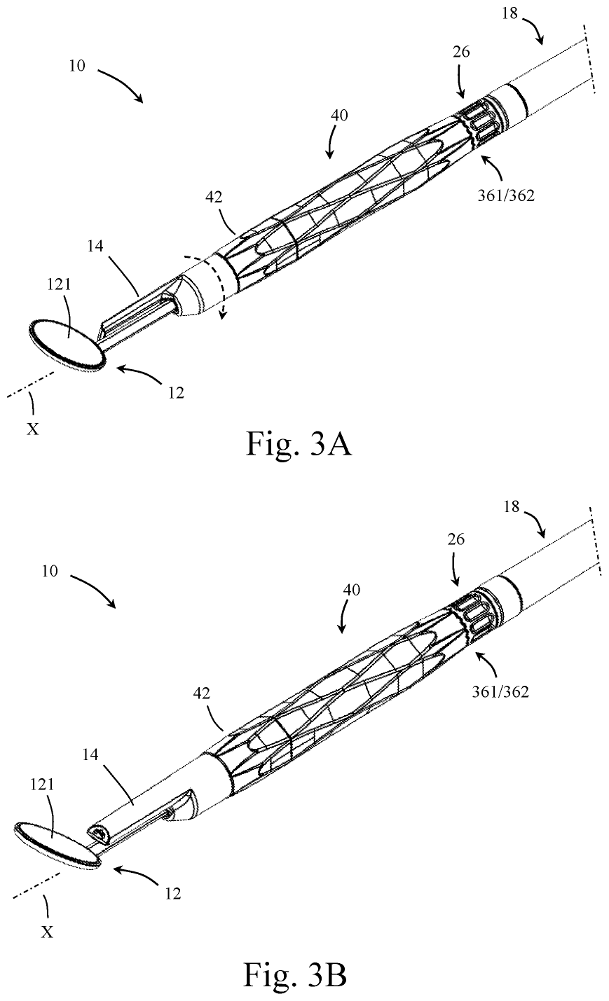

Attention is drawn to FIGS. 3A and 3B illustrating an embodiment of dental mirror system 10 in airflow miss-aligned (FIG. 3A) and aligned (FIG. 3B) states. Such alignment may be defined as axial alignment between an airflow emitted/ejected from airflow nozzle 14 and a reflecting surface 121 (possibly generally a center of surface 121) of a mirror head attached to system 10.

A dentist connecting a standardized mirror head to system 10 accordingly inserts a screw thread 142 of the mirror head into the threaded internal passage 381 of insert 38, which is accordingly preferably integrally fitted within shaft 40 that is molded thereupon.

When screwing a minor head into threaded passage 381, one may not necessarily know at what rotational angle the mirror head may end up being—when it is firmly fully screwed into passage 381 and gripped therein. This may be due, inter alia, to threads being formed on stems of such mirrors heads—starting at an arbitrary angle about an axis of the stem—for each mirror head being produced.

Thus, it may very likely be that the reflecting surface 121 of the mirror head may end up being miss-aligned with airflow nozzle 14 as illustrated in FIG. 3A—possibly each time a new mirror head is connected to the system.

According to an aspect of the present invention, after firmly attaching a mirror head 12 to an embodiment of a minor dental system 10—an alignment step may be performed between airflow nozzle 14 and the reflecting surface 121 of the mirror head—by manually rotating airflow nozzle 14 (as indicated by the dashed arrow in FIG. 3A) about axis X to an aligned state with reflecting surface 121 (as seen in FIG. 3B).

Such alignment may be required each time a new mirror head is screwed into passage 381 of the system. The frictional force applied by cylindrical flange 42 against airflow nozzle 14 may assist in making the alignment more controllable for a dentist—since he may be required to apply slight force in order to urge rotation to occur.

Further viewed in these figures, is that in the assembled state of the system's first and second portions 101, 102—their respective wavy shoulders 361, 362 mesh and engage each other. This meshing engagement determines that regulator head 26 of the first portion is maintained fixed against rotation relative to shaft 40 while handle 18 may be left able to rotate about axis X relative shaft 40.

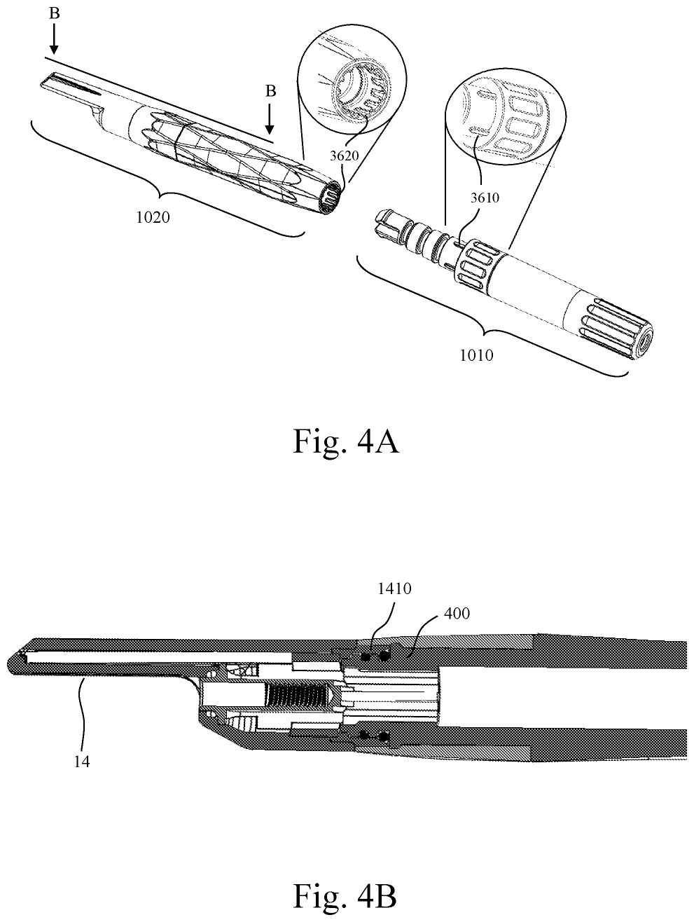

Attention is drawn to FIGS. 4A and 4B schematically illustrating a further embodiment of a dental mirror system, exemplifying e.g. another meshing arrangement that may be formed between the system's first and second portions here indicated by numerals 1010, 1020. Such meshing may be arranged to occur between a series of slits 3620 formed within the internal passage passing through distal second portion 1020 and corresponding bulges 3610 formed on a periphery of the forward coupler of proximal first portion 1010 that is arranged to enter the internal passage of second portion 1020 in the assembled state of the dental mirror system.

FIG. 4B, which is a view of a portion of distal second portion 1020 taken along cross section BB marked in FIG. 4A illustrates a possible manner of forming the frictional forces that control rotation of airflow nozzle. Here, a proximal shoulder 1410 of (or fixedly associated with) airflow flow nozzle 14 can be seen being arranged to press against shaft 400 of the distal portion 1020 in order to form such friction. Possibly, shoulder 1410 presses against O-rings located about shaft 400.

Attention is drawn back to FIGS. 3A and 3B. A dentist handling system 10 typically grasps with one hand shaft 40 and with his other hand handle 18. In an aspect of the present invention, a dentist may regulate the flow of air/gas passing through the system and emitted against a reflecting surface of a mirror head via airflow nozzle 14—by rotating handle 18 relative to shaft 40.

Since regulator head 26 is accordingly maintained fixed against rotation relative to shaft 40 by the meshing wavy shoulders 361, 362—also regulator base 20 is kept from rotating about axis X due to the meshing engagement of its arms 21 with the pegs 29 of head's anchor 31. The rotation of handle 18 relative to shaft 40 on the other hand may be translated to axial movement of regulator base 20 due to the threading engagement of the base's thread 17 in handle 18.

A rotation of handle 18 in a first rotational direction about axis X may be adapted to urge axial movement of regulator base 20 towards regulator head 26 while rotation of handle 18 in an opposing rotational direction about axis X may be adapted to urge axial movement of regulator base 20 away from regulator head 26.

Such axial movements of regulator base 20 away or towards regulator head 26 may be adapted to respectively urge pin 30 away or towards aperture 23 and by that assist in increasing or decreasing a route-way at that location for the flow of air/gas through the system towards airflow nozzle 14. Thus, by controlling the size/cross-section of this route-way—regulating the flow of air/gas emitted against a reflecting surface of a mirror head attached to the system may be accomplished.

In the description and claims of the present application, each of the verbs, “comprise” “include” and “have”, and conjugates thereof, are used to indicate that the object or objects of the verb are not necessarily a complete listing of members, components, elements or parts of the subject or subjects of the verb.

Further more, while the present application or technology has been illustrated and described in detail in the drawings and foregoing description, such illustration and description are to be considered illustrative or exemplary and non-restrictive; the technology is thus not limited to the disclosed embodiments. Variations to the disclosed embodiments can be understood and effected by those skilled in the art and practicing the claimed technology, from a study of the drawings, the technology, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures can not be used to advantage.

The present technology is also understood to encompass the exact terms, features, numerical values or ranges etc., if in here such terms, features, numerical values or ranges etc. are referred to in connection with terms such as “about, ca., substantially, generally, at least” etc. In other words, “about 3” shall also comprise “3” or “substantially perpendicular” shall also comprise “perpendicular”. Any reference signs in the claims should not be considered as limiting the scope.

Although the present embodiments have been described to a certain degree of particularity, it should be understood that various alterations and modifications could be made without departing from the scope of the invention as hereinafter claimed.

1. A dental mirror system extending along an axis X and being arranged to detachably receive standardized mirror heads at its distal axial side where each such standardized mirror head comprises a threaded member with a thread that starts at an arbitrary angle about its axis, the system comprising a threaded internal passage and an airflow nozzle, the threaded internal passage extending along axis X and being arranged to receive each time a threaded member of one standardized mirror head and the airflow nozzle being rotatable about the axis X in order to permit alignment of airflow emitted by the nozzle against a reflecting surface of the standardized mirror head.

2. The dental mirror system of claim 1 and comprising an insert, preferably made of metallic material, and the threaded internal passage is comprised in the insert, wherein possibly at least a portion of the system is molded over the insert.

3. The dental mirror system of claim 2, wherein the threaded internal passage is open to a distal side of the system.

4. The dental mirror system of claim 3 and being coupled at a proximal side to a pressurized air/gas source.

5. The dental mirror system of claim 4, wherein alignment of airflow emitted by the nozzle comprises rotating the airflow nozzle about the system's axis relative to other portions of the system.

6. The dental mirror system of claim 5, wherein rotating the airflow nozzle is against friction formed between the airflow nozzle and another portion of the system.

7. The dental mirror system of claim 1 and comprising a handle and a shaft and the handle being rotatable relative to the shaft in order to regulate the airflow emitted by the nozzle.

8. The dental mirror system of claim 7 and comprising a regulating member located within the handle and rotating the handle comprises axially moving the regulating member.

9. The dental mirror system of claim 8, wherein the airflow nozzle is coupled to the shaft.

10. The dental mirror system of claim 9, wherein the threaded internal passage is located within the shaft.

11. A method for aligning an airflow to a dental mirror comprising the steps of:
providing a mirror dental system extending along an axis X and comprising a threaded internal passage extending along the axis X and an airflow nozzle that is rotatable about the axis X,
attaching a standardized mirror head to the system by screwing a threaded stem of the mirror head into the axially extending internal passage of the dental system until it is firmly secured therein, wherein a thread on a threaded member of a standardized mirror head starts at an arbitrary angle about the threaded member's axis, and
rotating the airflow nozzle about the system's axis X in order to align an airflow emitted by the nozzle against a reflecting surface of the mirror head.

12. The method of claim 11 and comprising an insert, preferably made of metallic material, and the threaded internal passage is comprised in the insert.

13. The method of claim 12, wherein the threaded internal passage is open to a distal side of the system.

14. The method of claim 13 and being coupled at a proximal side to a pressurized air/gas source.

15. The method of claim 14, wherein alignment of airflow emitted by the nozzle comprises rotating the airflow nozzle about the system's axis relative to other portions of the system.

16. A method for regulating an airflow against a dental mirror comprising the steps of:
providing a dental mirror system having an axis and comprising a handle, a shaft located axially distal to the handle and an airflow passage extending axially through the handle and shaft, wherein the handle comprises a regulating member and the shaft being arranged for attachment of mirror heads at its distal axial side, wherein
rotating the handle relative to the shaft is arranged to regulate the flow of air/gas passing through the system and emitted against a reflecting surface of a mirror head attached to the system.

17. The method of claim 16, wherein rotating the handle comprises moving the regulating member along the system's axis.

18. (canceled)
